(19) United States
(12) Patent Application Publication
(10) Pub. No.: US 2025/0251393 A1
(43) Pub. Date: Aug. 7, 2025

(54) APOB:CFAS CONTENT TO AID IN THE DIAGNOSIS AND TREATMENT OF CARDIOVASCULAR DISEASES

(71) Applicant: Mohamed Zayed, St. Louis, MO (US)

(72) Inventor: Mohamed Zayed, St. Louis, MO (US)

(73) Assignee: Washington University, St. Louis, MO (US)

(21) Appl. No.: 19/047,329

(22) Filed: Feb. 6, 2025

Related U.S. Application Data
(60) Provisional application No. 63/550,784, filed on Feb. 7, 2024.

(51) Int. Cl. G01N 33/541; G01N 33/573; G01N 33/68; G01N 33/92

(52) U.S. Cl. CPC G01N 33/541; G01N 33/573; G01N 33/6893; G01N 33/92; G01N 2333/775; G01N 2333/91057; G01N 2800/042; G01N 2800/323; G01N 2800/52

(57) ABSTRACT

Methods of circulating Fatty Acid Synthase (cFAS) detection, assays, and kits thereof, as well as methods of diagnosis and treatment for cardiovascular-related diseases based on cFAS are provided. Exemplary embodiments of the methods, assays, and kits disclosed herein allow for improved, noninvasive, low-cost, and reliable diagnosis and treatment for cardiovascular-related diseases, particularly in subjects suffering from both symptomatic and asymptomatic Peripheral Arterial Disease (PAD), via detecting and measuring cFAS from LDL-associated cFAS captured from a biological sample.

### CROSS-REFERENCE TO RELATED APPLICATIONS

This application claims the benefit of priority to U.S. Provisional Application Ser. No. 63/550,784 filed on Feb. 7, 2024, which is incorporated herein by reference in its entirety.

### STATEMENTS REGARDING FEDERALLY SPONSORED RESEARCH OR DEVELOPMENT

This invention was made with government support under HL153262 awarded by the National Institutes of Health. The government has certain rights in the invention.

### MATERIAL INCORPORATED-BY-REFERENCE

Not applicable.

### FIELD

The present disclosure generally relates to methods for diagnosis and treatment of cardiovascular diseases, including serum tests for biomarker monitoring.

### BACKGROUND

Over the last two decades peripheral arterial disease (PAD) has become a national and international health concern that affects over 12 million individuals in the United States and more than 200 million individuals worldwide. More than 70% of individuals with PAD are asymptomatic or with atypical symptoms. Delayed diagnosis of PAD in asymptomatic individuals can lead to disease progression and the manifestation of chronic limb threatening ischemia (CLTI).

More than 40% of individuals with asymptomatic PAD eventually progress to symptomatic status. The Reduction of Atherothrombosis for Continued Health (REACH) Registry, an international prospective cohort of >68,000 individuals, provided high-quality data demonstrating that individuals with asymptomatic PAD are unfortunately commonly underdiagnosed and undertreated. Similarly, the multicenter PARTNERS program of 350 primary care practices, demonstrated that although prevalence of PAD is high, physician and patient awareness of the underlying disease remains very low. Since nearly 60% of individuals with PAD also have poly-vascular disease (coronary artery and/or cerebrovascular), missing a PAD diagnosis also puts the patient at higher risk of ischemic cardiac events, stroke, and death. Indeed, it is estimated that early diagnosis of asymptomatic PAD, can improve outcomes in at least 1 in 5 men and 1 in 3 women, who would otherwise not receive intensive medical management (such as with lipid lower therapy) as recommended by the American College of Cardiology (ACC) and Adult Treatment Panel (ATP) III guidelines.

The current diagnostic assessments of PAD have limitations that hinder patient diagnosis. The Framingham Risk Score (FRS) is often considered the reference standard for assessing risk of cardiovascular disease. However, by itself the scoring mechanism has limited accuracy since it tends to over-estimate risk in low-risk populations and underestimate risk in high-risk populations. The incorporation of other risk markers, such as metabolic syndrome and plasma C-reactive protein (C-RP) has had minimal success in improving the diagnosis of PAD. Accordingly, the 2016 American Heart Association (AHA) and ACC guidelines recommend diagnosis of PAD with either a walking impairment severity assessment (Class IB recommendation), or by obtaining blood pressure measurements to obtain an ankle-brachial index (ABI; Class IB). While these assessments can identify patients with classical PAD symptoms, they are unfortunately not generalizable for the majority of asymptomatic patients (Table 1). As such, ABI testing alone has demonstrated a limited sensitivity for PAD in the general population. Alternatives to the ABI include toe-brachial index (TBI), transcutaneous oxygen pressure (TcPO₂), skin perfusion pressure (SPP), or arterial duplex. While these tests are all noninvasive tools, they all require sophisticated technical equipment, and specialized training to perform procedures and interpret the results. Computed tomography angiography (CTA) and magnetic resonance angiography (MRA) are also useful to diagnose anatomic locations of arterial obstruction, but are poor primary diagnostic tools given their high costs. Thus, a serum immunoassay with high sensitivity and specificity is needed to reliably diagnose individuals with PAD.

### SUMMARY

Among the various aspects of the present disclosure are the provisions of detection and measurement of circulating Fatty Acid Synthase (cFAS) via LDL-associated (including ApoB-associated) cFAS, as well as diagnoses and treatment of cardiovascular and cardiovascular-related diseases.

In accordance with one aspect of the present disclosure, a method of detecting a level of circulating Fatty Acid Synthase (cFAS) in a subject is provided. The method comprises: providing a biological sample from the subject, immunocapturing low-density lipoprotein (LDL)-associated cFAS (LDL: cFAS) from the biological sample; and detecting the level of cFAS based on the immunocaptured LDL: cFAS.

In some embodiments, the biological sample is selected from blood, serum, and plasma. In some embodiments, immunocapturing of the LDL: cFAS comprises co-immunoprecipitating Apolipoprotein B (ApoB)-associated cFAS (ApoB: cFAS), and detecting the level of cFAS is based on the co-immunoprecipitated ApoB: cFAS. In some embodiments, detecting the level of cFAS content does not comprise measuring a gene that encodes a FAS enzyme.

In accordance with an additional aspect of the present disclosure, an immunoassay kit for detection of circulating Fatty Acid Synthase (cFAS) is provided. The kit comprises: a serum immunoassay which includes a capture antibody for ApoB, and a detection antibody for cFAS;

In some embodiments, the immunoassay kit further comprises one or more additional kit components. In some embodiments, the serum immunoassay does not detect cFAS by measuring a gene that encodes a FAS enzyme.

In accordance with a further aspect of the present disclosure, a method of treating a subject having at least one cardiovascular risk factor or cardiovascular-related condition is provided. The method comprises: providing a biological sample from the subject; co-immunocapturing low-density lipoprotein (LDL)-associated circulating Fatty Acid Synthase (cFAS) (LDL: cFAS) from the biological sample; measuring the level of cFAS based on the immunocaptured LDL: cFAS; and treating the subject with a therapeutic agent based on the detected level of cFAS.

In some embodiments, the biological sample is selected from blood, serum, and plasma. In some embodiments, the subject has at least one cardiovascular risk factor selected from diabetes, hypertension, hyperlipidemia, and smoking. In some embodiments, the subject has at least one cardiovascular-related condition selected from peripheral arterial disease (PAD), a foot wound, carotid occlusive disease, arteriosclerotic disease, atherosclerosis, carotid artery stenosis, and neurovascular disease. In some embodiments, the subject does not have cancer. In some embodiments, the immunocapturing of the LDL: cFAS comprises co-immunoprecipitating Apolipoprotein B (ApoB)-associated cFAS (ApoB: cFAS), and measuring the level of cFAS based on the co-immunoprecipitated ApoB: cFAS. In some embodiments, an elevated level of cFAS in the biological sample compared to a control indicates one of (i) increased levels of serum low-density lipoprotein (LDL) particles and (ii) increased levels of LDL particles deposited in high amounts in peripheral arterial plaque. In some embodiments, an elevated level of cFAS in the biological sample compared to a control indicates the subject is at risk for at least one cardiovascular-related condition selected from peripheral arterial disease (PAD), a foot wound, carotid occlusive disease, arteriosclerotic disease, atherosclerosis, carotid artery stenosis, and neurovascular disease. In some embodiments, an elevated level of cFAS in the biological sample compared to a control indicates the subject has at least one of type 2 diabetes, increased probability of scoring higher on a Diabetes Complications Severity Index (DCSI), and increased probability of having peripheral arterial disease (PAD). In some embodiments, measuring the level of cFAS comprises measuring a first level of cFAS and subsequently measuring a second level of cFAS, such that (i) an elevated first level of cFAS compared to the second level of cFAS indicates a decrease in disease severity or disease progression; and (ii) an elevated second level of cFAS compared to the first level of cFAS indicates an increase in disease severity or disease progression. In some embodiments, treating the subject comprises continuing treatment with the therapeutic agent when the second level of cFAS is elevated compared to the first level of cFAS, wherein continuing treatment comprises maintaining or increasing a dosage of the therapeutic agent. In some embodiments, treating the subject comprises continuing treatment with the therapeutic agent when the first level of cFAS is elevated compared to the second level of cFAS, wherein continuing treatment comprises maintaining or decreasing a dosage of the therapeutic agent. In some embodiments, treating the subject comprises ceasing treatment with the therapeutic agent when the first level of cFAS is elevated compared to the second level of cFAS.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is an illustration of peripheral arterial disease (PAD) severity of an artery in a leg. As peripheral arteries develop stenosis and obstructions, individuals with PAD progress from a compensated asymptomatic status to a symptomatic critically ischemic status.

FIG. 2A is an image of a lower leg x-ray of a patient with severe end-stage PAD and chronic limb threatening ischemia (CLTI). The arrows show severe calcific plaques extending in the tibial arteries.

FIG. 2B is an image of an angiogram in the lower leg of a patient with severe end-stage PAD and CLTI. The arrows point to areas of severe multivessel tibial vessel occlusive disease.

FIG. 2C is an image of the lower leg of a patient with severe end-stage PAD and CLTI. The calcaneal eschar is consistent with severe tissue loss.

FIG. 3A is a graph of circulating fatty acid synthase (cFAS) levels in VLDL, LDL, and HDL fractioned by fast-protein liquid chromatography (FPLC)

FIG. 3B is an image of a western blot of FAS and Apolipoprotein B (ApoB) co-immunoprecipitated from LDL cholesterol fractions isolated from a patient with type 2 diabetes (T2D).

FIG. 3C is a representative electron microscopy (EM) image of gold labeled cFAS (black oval) associated with LDL particles (light gray ovals).

FIG. 3D is a representative image of a western blot for cFAS and GAPDH control from serum of wildtype (WT; +/+), diabetic (db/db), cFAS selective knockdown in liver (fasL−/−), and cFAS selective knockdown in skeletal muscle (fasM−/−). n=6 per group.

FIG. 3E is a graph quantifying the western blot in FIG. 3D. The graph shows the amount of cFAS in the serum of +/+, db/db, fasL−/−, and fasM−/−mice. n=6 per group. Error bars, SEM, **p<0.01.

FIG. 4A is a graph of serum cFAS obtained from a fasting blood sample from individuals with no PAD (n=34), asymptomatic PAD (n=85), symptomatic PAD (n=142), and CLTI (n=173). Error bars, SEM. ***p<0.001.

FIG. 4B is a graph of serum LDL obtained from a fasting blood sample from individuals with no PAD (n=34), asymptomatic PAD (n=85), symptomatic PAD (n=142), and CLTI (n=173). Error bars, SEM. ***p<0.001.

FIG. 5A is a graph of multivariable logistic regression showing the associated between cFAS and CLTI compared to a reference line (black diagonal line) representing random probability. Serum cFAS content was evaluated using Aviva ELISA in patients with CLTI. cFAS content predicted the presence of CLTI with high accuracy (blue line; Hosmer-Lemeshow and C-index goodness-of-fit tests; area under ROC [AUROC] 0.83 [0.74-0.92]; P<0.001). Association between cFAS and CLTI remained significant when the cohort was adjusted for age and sex (red line; AUROC 0.84 [0.76-0.93]).

FIG. 5B is a graph of liner regression showing the positive correlation between serum cFAS and intraluminal plaque FAS content. Plaque cFAS content was evaluated using Aviva ELISA in patients with CLTI (R²=0.22; p=0.02).

FIG. 6A is a graph of serum cFAS content in patients with severe arterial atherosclerosis (n=26) and no atherosclerosis (n=13).

FIG. 6B is a graph showing no correlation between cFAS levels and serum glucose levels (R²=−0.02).

FIG. 6C is a graph showing no correlation between cFAS levels and serum LDL levels (R²=−0.5).

FIG. 6D is a graph showing no correlation between cFAS levels and total cholesterol levels (R²=−0.26).

FIG. 7 is a chart summarizing the superiority of profiling serum cFAS to diagnose PAD compared to other testing methods. Profiling cFAS does not require technical expertise, is serum based, not impacted by statins or glycemic status, and can detect asymptomatic and symptomatic PAD.

FIG. 8 is a schematic of the AirSeal diagnostic test to detect cFAS in human serum. A capture antibody (Ab) specific for C-terminus peptide sequence of ApoB is pre-coated onto a microwell. A serum sample is then added to the wells. A biotinylated detector Ab specific for N-terminus peptide sequence of cFAS is then added. After a brief incubation and wash, a Streptavidin-HRP conjugate is then added, and remaining unbound conjugate is washed off. The conjugate reaction is then facilitated by addition of a tetramethylbenzidine (TMB) substrate. A blue to yellow color change then occurs after addition of a TMB stop solution, and optical absorbance at 450 nm is quantified.

### DETAILED DESCRIPTION

The present disclosure is based, at least in part, on the discovery of novel and non-obvious methods of detecting and measuring serum cFAS, thus enabling diagnoses and treatments based on subject cFAS levels.

The present disclosure provides for the detection and measurement of circulating Fatty Acid Synthase (cFAS) for diagnosing and monitoring of disease, as well as for directing treatment. For example, cFAS in blood includes serum and/or plasma cFAS. Unlike conventional biomarkers, cFAS detection as described herein is detected by association with LDL (or a component thereof such as ApoB), not merely as the presence of a known gene encoding FAS or as free, un-associated cFAS. Furthermore, FAS (an essential multiunit enzyme that catalyzes the synthesis of long-chain fatty acids from acetyl-CoA and malonyl-CoA) was only recently found to circulate in the plasma. As such, detection of cFAS, and particularly LDL- or ApoB-associated cFAS, is unconventional.

### Cardiovascular-Related Disease, Disorder, or Condition

As described herein, the present disclosure provides for a new biomarker of cardiovascular disease. For example, the present disclosure showed that cFAS is associated with cardiovascular-related diseases, disorders, or conditions.

A cardiovascular-related disease can be any disease related to a disease, disorder, or condition of the heart and vasculature. For example, a cardiovascular-related disease can be aneurysm, angina, arrhythmia, arteriosclerotic disease, atherosclerosis, atrial fibrillation, cardiomyopathy, cardiovascular disease (CVD), cardiovascular mortality, cerebral vascular disease, cerebrovascular heart disease, congenital heart disease, coronary heart disease (CHD), coronary artery disease, heart attack, heart disease, heart failure (HF), heart valve disease, heart valve problems, high blood pressure (hypertension), hypertensive heart disease, infective endocarditis, inflammatory heart disease, ischemic heart disease, rheumatic heart disease, stroke (e.g., ischemic stroke, hemorrhagic stroke), sudden death, transient ischemic attack, peripheral arterial disease, pericardial disease, or valvular heart disease.

In one embodiment, the cardiovascular-related disease can be atherosclerosis. Atherosclerosis (e.g., peripheral arterial disease (PAD), diabetes-induced PAD, hypertension, arterial stenosis, carotid artery disease) is the hardening and narrowing of the arteries, which blocks the arteries putting blood flow at risk. Atherosclerosis is implicated as the cause of heart attacks, strokes, and peripheral vascular disease—what together are called cardiovascular disease.

### Therapeutic Agent

As described herein, a subject having a cardiovascular risk factor or cardiovascular-related disease can be treated by any method known in the art suitable for treating the disease, disorder, or condition. Therapeutic agents and methods of treating a cardiovascular-related disease, disorder, or condition are well known in the art.

For example, a therapeutic agent can be any therapeutic agent suitable for treating a cardiovascular-related disease, disorder, or condition or any agent suitable to avoid cardiovascular mortality or non-cardiovascular mortality.

As another example, a therapeutic agent can be a phosphodiesterase inhibitor (e.g., cilostazol, a vasodilator), an ACE inhibitor (e.g., a vasodilator, which opens blood vessels more fully and can help reduce high blood and slow heart failure), an anti-arrhythmic medication (helps restore a normal pumping rhythm to the heart), antibiotics (help to prevent the onset of infections), anticoagulants (“blood thinners” to reduce the risk of developing blood clots from poorly circulating blood around faulty heart valves), beta-blockers (can reduce the heart's workload by helping the heart beat slower, reduce palpitations), calcium channel blockers, diuretics (“water pills” to reduce amount of fluid in the tissues and bloodstream which can lessen the workload on the heart), vasodilators (can lower the heart's work by opening and relaxing the blood vessels; reduced pressure may encourage blood to flow in a forward direction, rather than being forced backward through a leaky valve), a thrombolytic agent, an anticonvulsant agent, an anti-platelet agent, an anti-coagulant agent or a hematologic agent, an analgesic, a beta blocker or alpha activity agent, an ACE inhibitor, a calcium channel blocker, a vasodilator, a cholesterol-lowering and blood-pressure-lowering medicine, a blood pressure medicine, or medicines used to treat depression and pain.

As another example, a therapeutic agent can be an anticoagulant such as Warfarin (for example, Coumadin, Jantoven), Heparin, Dabigatran (Pradaxa), Rivaroxaban (Xarelto), Apixaban (Eliquis), or Edoxaban (Savaysa). As another example, a therapeutic agent can be a thrombolytic such as an IV tissue plasminogen activator (TPA) or Alteplase (Activase). As another example, a therapeutic agent can be an antiplatelet medication such as aspirin (for example, Bayer) or aspirin combined with dipyridamole (Aggrenox), Clopidogrel (Plavix), Prasugrel (Effient), or Ticagrelor (Brilinta). As another example, a therapeutic agent can be a cholesterol-lowering and blood-pressure-lowering medicines such as a statin (e.g., Atorvastatin (Lipitor), Rosuvastatin (Crestor)), angiotensin II receptor blockers (ARBs), angiotensin-converting enzyme (ACE) inhibitors, beta-blockers, calcium channel blockers, diuretics, Nicotinic Acids (e.g., Lovastatin (Advicor)), Cholesterol Absorption Inhibitors (e.g., Ezetimibe/Simvastatin (Vytorin)). As another example, a therapeutic agent can be a diuretic such as Amiloride (Midamor), Bumetanide (Bumex), Chlorothiazide (Diuril), Chlorthalidone (Hygroton), Furosemide (Lasix), Hydro-chlorothiazide (Esidrix, Hydrodiuril), Indapamide (Lozol, or Spironolactone (Aldactone). As another example, a therapeutic agent can be a medicine used to treat depression and pain such as amitriptyline, bupropion (Wellbutrin), citalopram (Celexa), fluoxetine (Prozac), sertraline (Zoloft), venlafaxine (Effexor). As another example, a therapeutic agent can be an anticonvulsant such as Diazepam (Valium) or Lorazepam (Ativan). As another example, a therapeutic agent can be an analgesic such as acetaminophen (Tylenol, Feverall, Aspirin Free Anacin). As another example, a therapeutic agent can be a beta blocker or alpha activity medication such as Labetalol (Normodyne, Trandate), Acebutolol (Sectral), Atenolol (Tenormin), Betaxolol (Kerlone), Bisoprolol/hydrochlorothiazide (Ziac), Bisoprolol (Zebeta), Metoprolol (Lopressor, Toprol XL), Nadolol (Corgard), Propranolol (Inderal), or Sotalol (Betapace). As another example, a therapeutic agent can be a digitalis preparation (e.g., Digoxin, Digitoxin) such as Lanoxin. As another example, a therapeutic agent can be a combined alpha and beta blocker such as carvedilol or labetalol hydrochloride. As another example, a therapeutic agent can be an ACE Inhibitor such as Enalapril (Vasotec), Benazepril (Lotensin), Captopril (Capoten), Enalapril (Vasotec), Fosinopril (Monopril), Lisinopril (Prinivil, Zestril), Moexipril (Univasc), Perindopril (Aceon), Quinapril (Accupril), Ramipril (Altace), or Trandolapril (Mavik). As another example, a therapeutic agent can be a calcium channel blocker such as Nicardipine (Cardene), Amlodipine (Norvasc, Lotrel), Diltiazem (Cardizem, Tiazac), Felodipine (Plendil), Nifedipine (Adalat, Procardia), Nimodipine (Nimotop), Nisoldipine (Sular), or Verapamil (Calan, Verelan). As another example, a therapeutic agent can be a vasodilator such as Nitroprusside sodium (Nipride, Nitropress, Sodium Nitroprusside), Isosorbide dinitrate (Isordil), Nesiritide (Natrecor), Hydralazine (Apresoline), Nitrates, or Minoxidil. As another example, a therapeutic agent can be an Angiotensin-2 Receptor Blockers (ARBs) or Angiotensin-2 Receptor Antagonists such as Candesartan (Atacand), Eprosartan (Teveten), Irbesartan (Avapro), Losartan (Cozaar), Telmisartan (Micardis), or Valsartan (Diovan). As another example, a therapeutic agent can be an Angiotensin-Receptor Neprilysin Inhibitor (ARNI) (a new drug combination of a neprilysin inhibitor and an ARB) such as Sacubitril/valsartan (Entresto).

As another example, a therapeutic agent can be an agent that modulates FAS levels.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that replaces the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating a cardiovascular-related disease, disorder, or condition in a subject in need administration of a therapeutically effective amount of a therapeutic agent, so as to substantially inhibit a cardiovascular-related disease, disorder, or condition, slow the progress of a cardiovascular-related disease, disorder, or condition, or limit the development of a cardiovascular-related disease, disorder, or condition.

Methods described herein are generally performed on a subject in need thereof. A subject in need of the therapeutic methods described herein can be a subject having, diagnosed with, suspected of having, or at risk for developing a cardiovascular-related disease, disorder, or condition. A determination of the need for treatment is typically assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be a human subject.

Generally, a safe and effective amount of a therapeutic agent is, for example, an amount that would cause the desired therapeutic effect in a subject while minimizing undesired side effects. In various embodiments, an effective amount of a therapeutic agent described herein can treat or substantially inhibit a cardiovascular-related disease, disorder, or condition, slow the progress of a cardiovascular-related disease, disorder, or condition, or limit the development of a cardiovascular-related disease, disorder, or condition.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of a therapeutic agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to substantially inhibit a cardiovascular-related disease, disorder, or condition, slow the progress of a cardiovascular-related disease, disorder, or condition, or limit the development of a cardiovascular-related disease, disorder, or condition.

The amount of a composition described herein that can be combined with a pharmaceutically acceptable carrier to produce a single dosage form varies depending upon the host treated and the particular mode of administration. It is appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject depends upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, Mcgraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It is understood, however, that the total daily usage of the compounds and compositions of the present disclosure will be decided by an attending physician within the scope of sound medical judgment.

Again, each of the states, diseases, disorders, and conditions described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a therapeutic agent can occur as a single event or over a time course of treatment. For example, a therapeutic agent can be administered daily, weekly, bi-weekly, or monthly. For treatment of acute conditions, the time course of treatment is usually at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a cardiovascular-related disease, disorder, or condition.

A therapeutic agent can be administered simultaneously or sequentially with another agent, such as an antibiotic, an anti-inflammatory, or another agent. For example, a therapeutic agent can be administered simultaneously with another agent, such as an antibiotic or an anti-inflammatory. Simultaneous administration can occur through administration of separate compositions, each containing one or more of a therapeutic agent, an antibiotic, an anti-inflammatory, or another agent. Simultaneous administration can occur through administration of one composition containing two or more of a therapeutic agent, an antibiotic, an anti-inflammatory, or another agent. A therapeutic agent can be administered sequentially with an antibiotic, an anti-inflammatory, or another agent. For example, a therapeutic agent can be administered before or after administration of an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10:0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to capture antibodies, detection antibodies, and further reagents and/or supplies. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754; Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### Examples

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Peripheral Arterial Disease (PAD) Diagnosis Via Serum Circulating Fatty Acid Synthase (cFAS) Detection

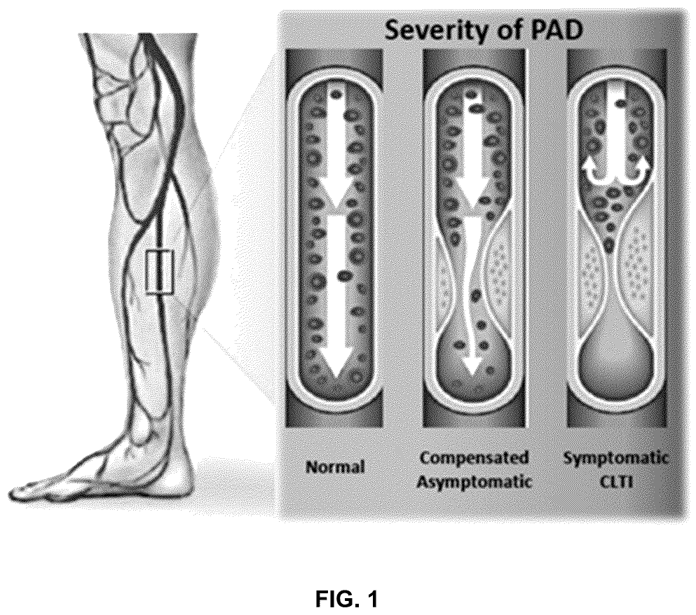

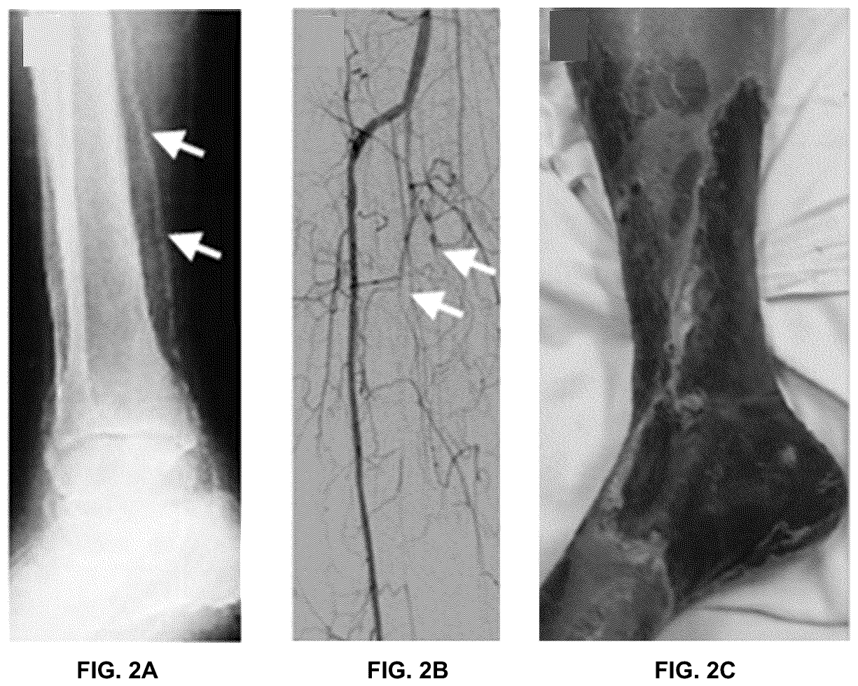

Peripheral Arterial Disease (PAD) impacts more than 200 million individuals worldwide, and >12 million in the U.S. (1 in 27 people). More than 95% of individuals with at least one cardiovascular risk factor (hyperlipidemia, diabetes, smoking), are currently living with PAD. Due to the aging population and growing prevalence of risk factors, prevalence of PAD is anticipated to rise by 25% over the next decade. However, over 50% of patients have compensated asymptomatic PAD (FIG. 1). Delayed diagnosis of PAD during this phase can lead to unchecked disease progression, rise of intermittent claudication (disabling extremity pain with exertion), and the eventual manifestation of chronic limb threatening ischemia (CLTI; FIG. 2A, FIG. 2B, FIG. 2C). Individuals with CLTI develop severe extremity pain, peripheral wounds, gangrene, and an increased risk of amputations. This leads to significant suffering, disability, higher mortality, and >$3.1 billion in yearly U.S. health care costs. The recent global vascular guidelines for the management of CLTI emphasized the need to develop methods for the early diagnosis of PAD prior to the onset of end-stage CLTI. Herein, a serum-based test has been developed to aid in the early diagnosis of PAD.

The current diagnostic assessments for PAD have limitations that hinder patient diagnosis (Table 1) and there are currently no FDA-approved blood/serum tests to aid in the diagnosis of PAD. The AHA/ACC and the Society for Vascular Surgery (SVS) guidelines acknowledge this persistent clinical gap. Instead, Medicare-reimbursed lipid panel testing (serum total cholesterol, LDL, HDL, and triglyceride) and glycemic tests (Hemoglobin A1C; HbA1c) are recommended for secondary cardiovascular disease prevention. Indeed, routine lipid assessments in patients with PAD have been shown to guide medical therapy and reduce the incidence of ischemic coronary events. However, a Cochrane review of >10,000 individuals with PAD demonstrated no evidence that routine serum lipid assessments reduce PAD progression or improve rates of limb salvage. Similarly, multiple studies have evaluated whether serum lipid biomarkers such as LDL, HDL, triglycerides (TG), and Apolipoprotein A-1, have adequate sensitivity for the diagnosis for PAD (Table 2). Weak associations between serum lipid biomarkers and PAD have been identified, and there are very limited studies linking conventional serum lipid biomarkers to asymptomatic PAD. Therefore, there continues to be an unmet need for an accurate serum test to aid in the diagnosis and management of the earlier stages of PAD.

TABLE 1
Gaps in Diagnostic Assessments of PAD

| | |
|---|---|
| 1 | Non-generalizable tests for patients with either asymptomatic or |
| | symptomatic PAD |
| 2 | Serum lipids that have limited utility in asymptomatic patients and |
| | those taking lipid lowering medications |
| 3 | Procedural tests that require a high level of technical expertise |
| 4 | Procedural tests that have limited utility in unsuspecting |
| | asymptomatic patients with PAD |

TABLE 2
Summary of Clinical Studies of PAD Diagnostics

| | | | Lipids | OR |
|---|---|---|---|---|
| Study | Sex | PAD Status | Eval | (95% CI) |
| Framingham Study | ♂♀ | Claudication | TC | 1.2 (1.1-1.3) |
| Framingham Offspring | ♂♀ | Claudication | HDL | 0.9 (0.8-1.0) |
| Edinburgh Artery Study | ♂♀ | Claudication | TG | NS |
| Physicians' Health Study | ♂ | Claudication | HDL | 3.9 (1.7-8.6) |
| Women's Health Study | ♀ | Claudication | HDL | 0.4 (0.3-0.8) |
| Health Prof Follow-Up | ♂ | Surgical | TC | 1.4 (1.1-1.7) |
| Study | | Intervention | | |
| Speedwell Study | ♂ | Claudication | TG | 0.3 (0.2-0.5)* |

NS, Non-significant;
*Adjusted for age and smoking

### Results

Circulating Fatty Acid Synthase (cFAS) is ApoB-Associated and Originates from the Liver.

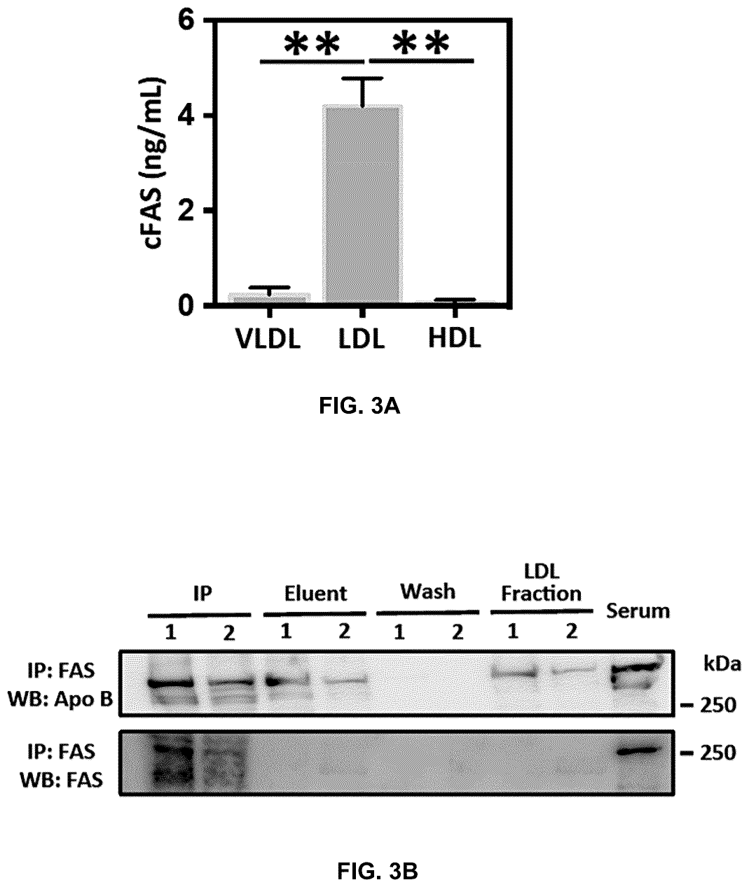

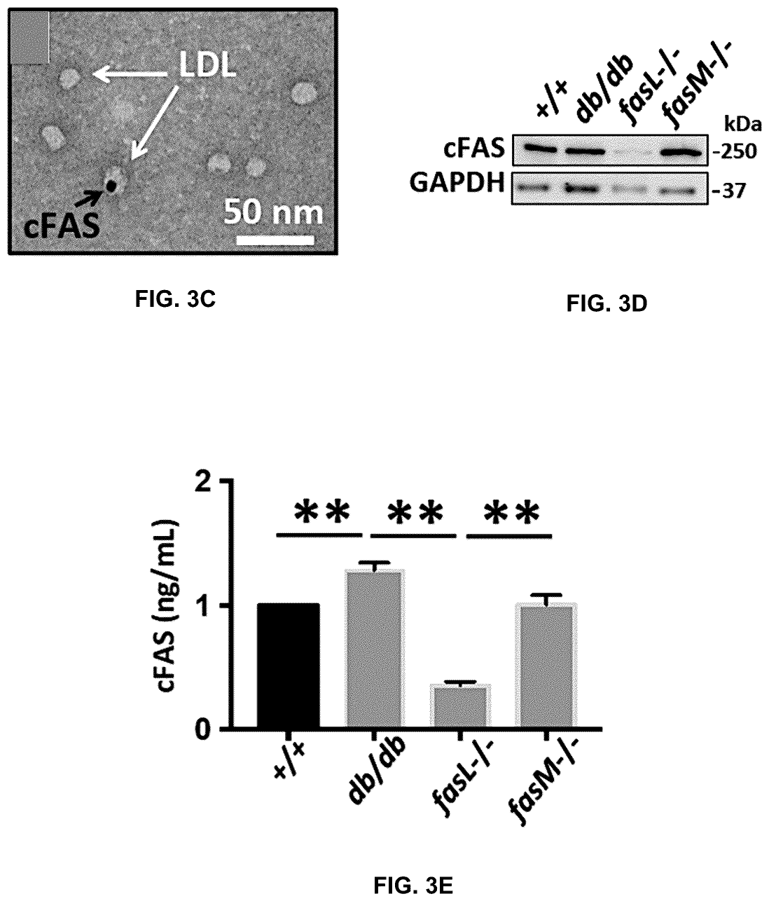

As discovered herein, cFAS, which is essential for de novo saturated fatty acid synthesis, almost exclusively fractionates in fast-protein liquid chromatography (FPLC)-derived LDL cholesterol isolates (P<0.01; FIG. 3A). Co-immunoprecipitation pull-down demonstrated that cFAS is associated with Apolipoprotein B (ApoB; the dominant protein in LDL particles; FIG. 3B). Gold-labeled cFAS is observed on LDL particles using electron microscopy (EM; FIG. 3C). Lastly, selective knockdown of FASN (gene expressing cFAS) in liver or skeletal muscle tissue, demonstrated that the liver is the dominant source of cFAS (P<0.01; FIG. 3D, FIG. 3E). Mice with a diabetes-like phenotype (db/db) have higher levels of cFAS. The link between cFAS and liver-derived LDL highlights the key metabolic role that cFAS may be playing in peripheral arterial atheroprogression. These findings support using a ApoB: cFAS immuno-capture strategy to detect cFAS in human serum.

cFAS is Elevated in Patients with PAD.

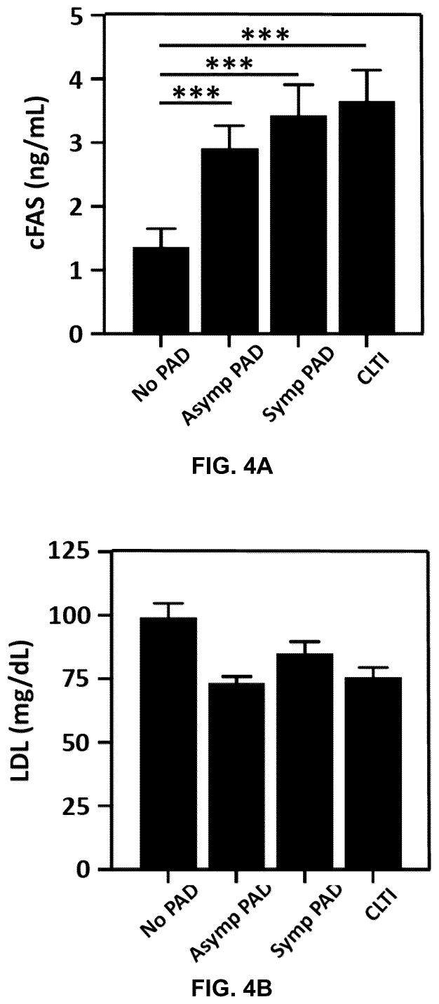

As herein discovered, cFAS is a biomarker for atherosclerotic disease severity in patients with PAD. To extend these findings, a trial was conducted on 434 individuals who voluntarily donated fasting serum samples to the Zayed laboratory biobank. Fasting serum specimens were collected from individuals with no PAD or cardiovascular risk factors, asymptomatic PAD (abnormal ABI of <0.9 but no symptoms of claudication or rest pain), symptomatic PAD (abnormal ABI and symptoms consistent with claudication), and CLTI (abnormal ABI and evidence of either rest pain, tissue loss, or foot/leg gangrene). Serum cFAS content was evaluated as a potential biomarker for PAD using ELISA. It was observed that cFAS serum content was dramatically elevated in all patients with PAD (asymptomatic and symptomatic; FIG. 4A). Interestingly, LDL was not significantly different between patients with and without PAD (FIG. 4B). This confirms that cFAS can be an important diagnostic test in individuals with asymptomatic or symptomatic PAD.

cFAS is Independently Associated with PAD.

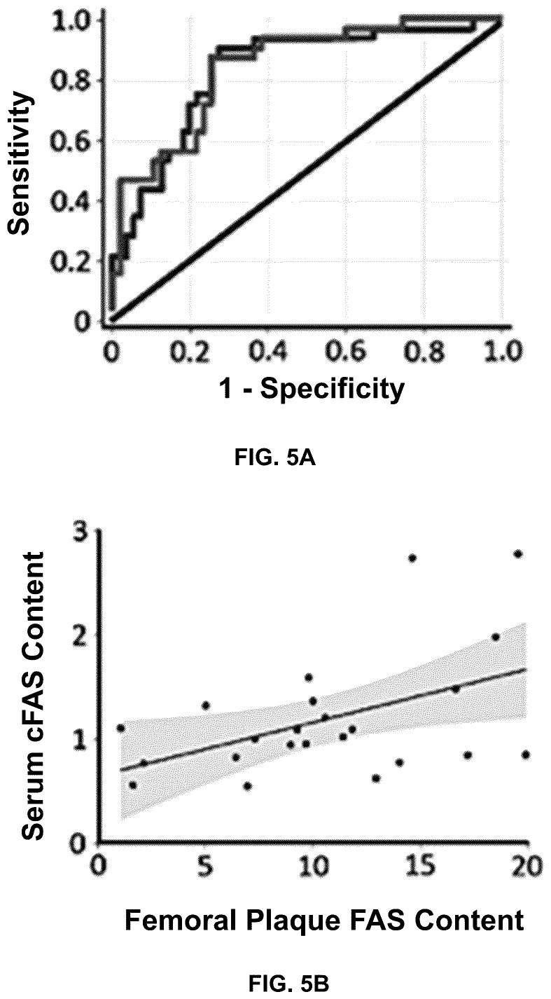

It was evaluated whether serum cFAS content can be used as an independent screen for CLTI (the most severe form of PAD). When adjusting for patient age and sex, it was observed that in patients with and without CLTI cFAS content were highly associated with CLTI (OR 1.17[1.04-1.31], P<0.001) with an 84% accuracy (area under ROC is 0.84 [0.74-0.92]; FIG. 5A). In this cohort, each unit of increase in cFAS increased the odds of CLTI by 16%. In a subset of 23 patients who also underwent femoral endarterectomy at time of arterial revascularization, linear regression analysis demonstrated a significant positive correlation between serum cFAS and plaque FAS (R2=0.22; P=0.02; FIG. 5B). These findings support serum cFAS as a diagnostic test for PAD.

TABLE 3
Summary of Superiority Profile of cFAS Serum Diagnostic Testing Compared
to Other PAD Testing Methods

| | | | | Un- | | |
|---|---|---|---|---|---|---|
| | No | | | Impacted | | |
| | Technical | | Un- | by | | |
| Diagnostic | Expertise | Serum- | Impacted | Glycemic | Asymptomatic | Symptomatic |
| Test | Required | Based | by Statins | Status | PAD | PAD |
| cFAS | ✓ | ✓ | ✓ | ✓ | ✓ | ✓ |
| Serum | ✓ | ✓ | [symbol] | ✓ | [symbol] | ✓ |
| Lipids | | | | | | |
| HbA1c | ✓ | ✓ | ✓ | [symbol] | [symbol] | [symbol] |
| ABI/TBI | [symbol] | [symbol] | ✓ | [symbol] | ✓ | ✓ |
| Arterial | [symbol] | [symbol] | ✓ | [symbol] | [symbol] | ✓ |
| Duplex | | | | | | |
| CTA/MRA | [symbol] | [symbol] | ✓ | [symbol] | [symbol] | ✓ |

Relationship Between cFAS and LDL.

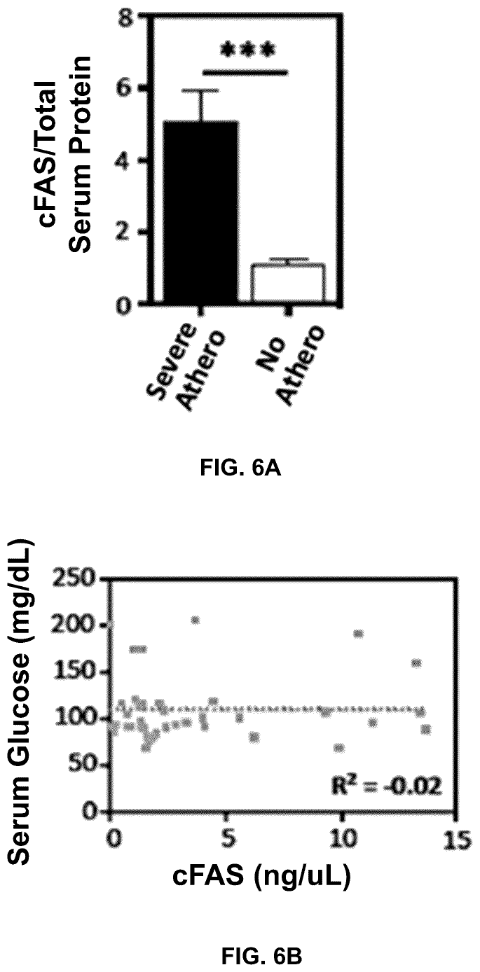

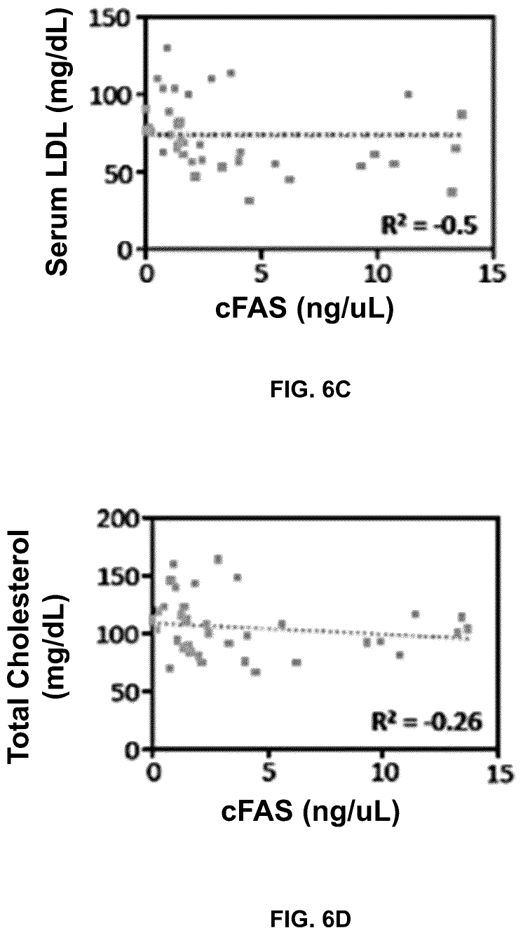

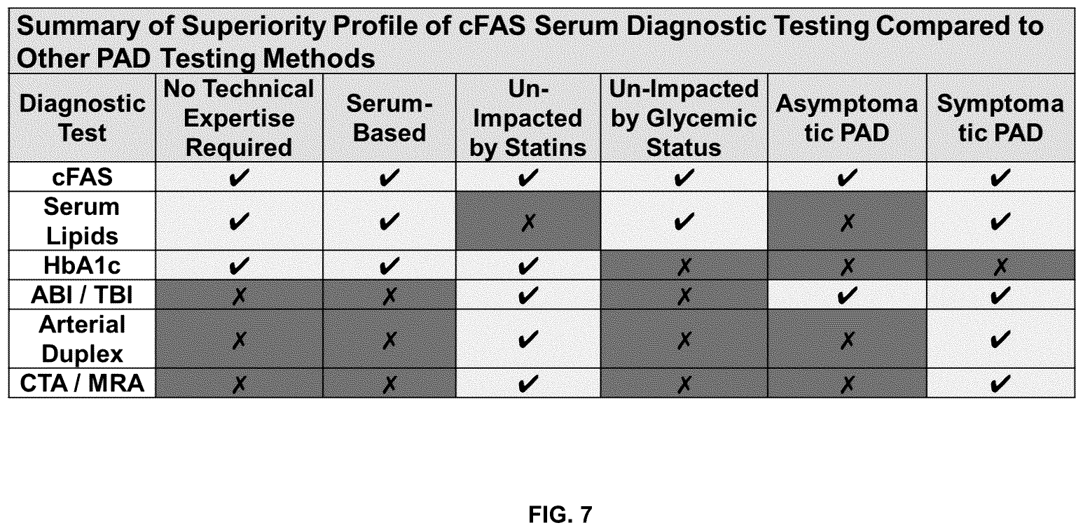

Serum cFAS content was evaluated in 26 patients with severe arterial atherosclerotic, and 13 healthy control patients with no atherosclerosis. Like patients with PAD and CLTI (FIG. 4A, FIG. 4B), significantly higher cFAS was observed in patients with arterial atherosclerosis (FIG. 6A). Serum cFAS content did not correlate with serum glucose, LDL, or total cholesterol (FIG. 6B, FIG. 6C, FIG. 6D). These data suggests that although cFAS is biochemically associated with LDL (FIG. 3C), its total serum content does not correlate with LDL. This observation supports that serum cFAS can be a superior independent diagnostic test for patients with PAD (Table 3).

Antibody Selection for Serum ApoB: cFAS Immunoassay Diagnostic Kit.

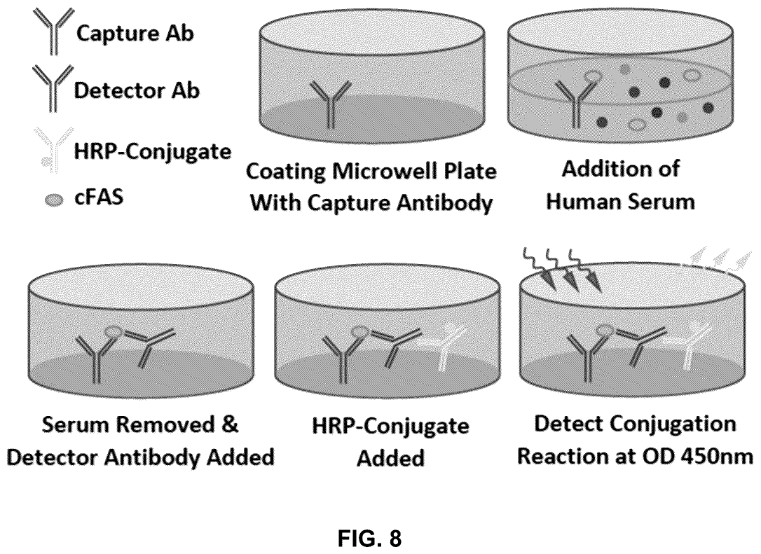

The present disclosure demonstrated that an immunoassay can accurately detect cFAS in murine and human serum (FIG. 3A, FIG. 3E, FIG. 4A, FIG. 5B), as confirmed through herein-disclosed and conventional methods (e.g., co-immunoprecipitation with ApoB, Western blotting, and electron microscopy; FIG. 3B, FIG. 3C, FIG. 3D). Human studies in >500 patients (FIG. 4A, FIG. 5B, FIG. 6A) were conducted using a cFAS ELISA (Aviva Biosystems, Inc.; product #OKCD08146) not approved for clinical diagnostic purposes. Antibody selection is required for a diagnostic immunoassay kit optimized for human serum testing. The disclosed immunoassay kit provides commercialization advantages, including low-cost of production, technical ease of use in a variety of clinical settings (including resource-limited outpatient settings in underserved communities), and rapid results that can enable timely clinical decision-making (further secondary screening versus initiation of intensive medical therapy). Exemplary embodiments of the diagnostic kit include an optimized capture antibody (to immobilize ApoB on a solid surface), and detection antibody (to quantify the immobilized cFAS; FIG. 8).

The ApoB capture antibody has high specificity targeting the C-terminus peptide sequence of ApoB. Specificity of this antibody to human ApoB in serum is evaluated with serum immunoprecipitation and 2D blotting studies (FIG. 3B). The biotinylated detector antibody separately binds to the N-terminus peptide sequence of human cFAS (sequence selected from known commercial antibody binding sites). The biotinylated detector antibodies are validated with recombinant scrambled (negative control) and non-scrambled (positive control) cFAS protein sequences. Binding affinity assays use commercially available streptavidin-horseradish peroxidase (HRP) or Avidin-HRP conjugation reaction to confirm the binding affinity of the ApoB capture antibody and biotinylated detector antibody. Linear dilutions of recombinant scrambled (negative control) and non-scrambled (positive control) cFAS sequences (0.1-50 ng/mL) are tested and quantified using a spectrophotometric absorbance at 450 nm to detect colorimetric change (blue to yellow; FIG. 8). Values for limit of detection (LOD) and limit of quantification (LOQ) are then derived to determine assay sensitivity. High binding affinity is determined by <0.5 ng/ml LOD and <30 ng/mL LOQ.

### Validation of Immunoassay Kit Sensitivity Using Biobanked Human Serum Samples.

The immunoassay kit enables detection of asymptomatic or symptomatic PAD with robust sensitivity (>90%). Data presented herein demonstrates that with a commercially available non-clinical research ELISA, serum cFAS content is elevated in individuals with asymptomatic PAD, symptomatic PAD, as well as CLTI (FIG. 4A, FIG. 5B). ApoB capture and cFAS detection immunoassay strategy was validated using already collected serum specimens from individuals with and without PAD.

Biobanked serum from over 1000 fasting patients were immediately processed and stored at −80C until further use. Patients recruited included ‘healthy’ patients (non-smoking and <40 years old), as well as patients with varying severities of PAD and Framingham Risk Scores (FRS). For all patients, 42 clinical variables (Framingham Risk Score, sex, age, medical co-morbidities, ABI, TBI, serum lipid metabolites, etc.) are collected and maintained in a secure RedCap database.

Serum from 400 biobank patients was analyzed to include an equal distribution of male and female patients, all ethnic groups (including Hispanic and Black individuals), and ages 40-89. Included patients comprise those with HbA1c between 5.5-11%, total serum cholesterol between 150-300 mg/dL, total serum LDL 70-200 mg/dL, and total serum TG 140-500 mg/dL. Patients with a prior/current cancer are excluded. Serum evaluation is from 4 distinct patient groups. Group 1:100 ‘healthy’ patients <40 years old with no cardiovascular risk factors; Group 2:100 patients with asymptomatic PAD (defined as individuals with ABI<0.9 but no self-reported claudication symptoms); Group 3:100 patients with symptomatic PAD (defined as individuals with abnormal ABI with claudication); Group 4:100 patients with CLTI (as defined by SVS guidelines, ABI<0.5, TBI<0.2, flat-line digital arterial waveform, rest pain, ischemic wounds, or tissue loss).

Total cFAS content, in some embodiments, is evaluated using the disclosed AirSeal immunoassay kit. For retrospective analysis, the null hypothesis is defined as cFAS content being the same between Groups 1 and 2. Based on the disclosed data (FIG. 4A, FIG. 5A, FIG. 5B), the alternative hypothesis estimates Group 1 mean (standard deviation; SEM)=1.4 (0.3), and Group 2 mean (SEM)=2.9 (0.4). With 100 patients in each group and a significance level of 5%, the power is ˜93.5% using a two-sided two-sample unequal-variance t-test. For adjusted analyses, additional covariates and combinations thereof are considered (Framingham Risk Score, sex, age, ethnicity, smoking status, serum LDL, etc.) for adjustment depending upon the embodiment. In some embodiments, in the adjusted multiple linear regression analysis model, assuming 1) number of variables to be adjusted is at least 7 (accounting for 30%, R²=0.3, of variation in serum cFAS content), and 2) group variable variation of an additional 10% (partial R² due to group=0.1), with 200 patients (Group 1 & 2) the estimated power is 88% (using an F test alpha=5%). Quality control for cFAS assessments is included to evaluate data reproducibility and variance. For each 25 patients a random repeat sample 10 is evaluated with both the disclosed AirSeal kit and Aviva ELISA, with a coefficient of variation (CV)<15% being considered reliable. In AirSeal immunoassay kit embodiments, a >90% sensitivity (<10% false negative rate) is achieved for detection of PAD in Group 2 (individuals with asymptomatic PAD) and PAD/CLTI in Groups 3 and 4.

What is claimed is:

1. A method of detecting a level of circulating Fatty Acid Synthase (cFAS) in a subject, the method comprising:
providing a biological sample from the subject,
immunocapturing low-density lipoprotein (LDL)-associated cFAS (LDL: cFAS) from the biological sample; and
detecting the level of cFAS based on the immunocaptured LDL: cFAS.

2. The method of claim 1, wherein the biological sample is selected from blood, serum, and plasma.

3. The method of claim 1, wherein
immunocapturing of the LDL: cFAS comprises co-immunoprecipitating Apolipoprotein B (ApoB)-associated cFAS (ApoB: cFAS), and
detecting the level of cFAS is based on the co-immunoprecipitated ApoB: cFAS.

4. The method of claim 1, wherein detecting the level of cFAS content does not comprise measuring a gene that encodes a FAS enzyme.

5. An immunoassay kit for detection of circulating Fatty Acid Synthase (cFAS) comprising:
a serum immunoassay including:
a capture antibody for ApoB, and
a detection antibody for cFAS.

6. The immunoassay kit of claim 5, further comprising one or more additional kit components.

7. The immunoassay kit of claim 5, wherein the serum immunoassay does not detect cFAS by measuring a gene that encodes a FAS enzyme.

8. A method of treating a subject having at least one cardiovascular risk factor or cardiovascular-related condition, the method comprising:
providing a biological sample from the subject;
immunocapturing low-density lipoprotein (LDL)-associated circulating Fatty Acid Synthase (cFAS) (LDL: cFAS) from the biological sample;
measuring a level of cFAS based on the immunocaptured LDL: cFAS; and
treating the subject with a therapeutic agent based on the measured level of CFAS.

9. The method of claim 8, wherein the biological sample is selected from blood, serum, and plasma.

10. The method of claim 8, wherein the subject has at least one cardiovascular risk factor selected from diabetes, hypertension, hyperlipidemia, and smoking.

11. The method of claim 8, wherein the subject has at least one cardiovascular-related condition selected from peripheral arterial disease (PAD), a foot wound, carotid occlusive disease, arteriosclerotic disease, atherosclerosis, carotid artery stenosis, and neurovascular disease.

12. The method of claim 8, wherein the subject does not have cancer.

13. The method of claim 8, wherein
immunocapturing of the LDL: cFAS comprises co-immunoprecipitating Apolipoprotein B (ApoB)-associated cFAS (ApoB: cFAS), and
measuring the level of cFAS is based on the co-immunoprecipitated ApoB: cFAS.

14. The method of claim 8, wherein an elevated level of cFAS in the biological sample compared to a control indicates one of:
increased levels of serum low-density lipoprotein (LDL) particles; and
increased levels of LDL particles deposited in high amounts in peripheral arterial plaque.

15. The method of claim 8, wherein an elevated level of cFAS in the biological sample compared to a control indicates the subject is at risk for at least one cardiovascular-related condition selected from peripheral arterial disease (PAD), a foot wound, carotid occlusive disease, arteriosclerotic disease, atherosclerosis, carotid artery stenosis, and neurovascular disease.

16. The method of claim 8, wherein an elevated level of cFAS in the biological sample compared to a control indicates the subject has at least one of type 2 diabetes, increased probability of scoring higher on a Diabetes Complications Severity Index (DCSI), and increased probability of having peripheral arterial disease (PAD).

17. The method of claim 8, wherein measuring the level of cFAS comprises measuring a first level of cFAS and subsequently measuring a second level of cFAS, such that
(i) an elevated first level of cFAS compared to the second level of cFAS indicates a decrease in disease severity or disease progression; and
(ii) an elevated second level of cFAS compared to the first level of cFAS indicates an increase in disease severity or disease progression.

18. The method of claim 17, wherein treating the subject comprises continuing treatment with the therapeutic agent when the second level of cFAS is elevated compared to the first level of cFAS, wherein continuing treatment comprises maintaining or increasing a dosage of the therapeutic agent.

19. The method of claim 17, wherein treating the subject comprises continuing treatment with the therapeutic agent when the first level of cFAS is elevated compared to the second level of cFAS, wherein continuing treatment comprises maintaining or decreasing a dosage of the therapeutic agent.

20. The method of claim 17, wherein treating the subject comprises ceasing treatment with the therapeutic agent when the first level of cFAS is elevated compared to the second level of cFAS.
